(19) United States
(12) Patent Application Publication
(10) Pub. No.: US 2005/0101899 A1
(43) Pub. Date: May 12, 2005

(54) Band aid applique

(76) Inventor: Anne Wegmann, St. Louis, MO (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 10/983,844

(22) Filed: Nov. 8, 2004

Related U.S. Application Data
(60) Provisional application No. 60/518,495, filed on Nov. 7, 2003.

(51) Int. Cl. A61F 13/00

(52) U.S. Cl. 602/41

(57) ABSTRACT

An appliqué sized to fit over a band aid. The appliqué has an adhesive backing which allows the band aid wearer to secure it over the top of the band aid. The outer surface of the appliqué has one of a number of decorative patterns formed on it so to give the band aid a pleasing appearance. The appliqué has removable layers of adhesive material so the appliqué can be used repeatedly when the band aid is changed. The appliqués are also made for use with other types of bandages such as eye patches.

### CROSS REFERENCE TO RELATED APPLICATIONS

Provisional patent application 60/518,495 filed Nov. 7, 2003.

### STATEMENT REGARDING FEDERALLY SPONSORED RESEARCH OR DEVELOPMENT

N/A

### BACKGROUND OF THE INVENTION

This invention relates to bandages such as band aids and the like; and, more particularly, to a decorative appliqué which can be placed over the band aid to make it more sightly and give it a pleasing, decorative appearance.

Bandages come in many varieties. The most common type is a band aid which is commercially available in a number of sizes and shapes to cover a wound and protect it while healing. Most band aids are rectangular in shape, an inch or so wide, and three to four inches long. These are typically used to cover small puncture wounds or cuts. Larger band aids are also available to cover large skins or scrapes. In most instances, the wound being protected heals rather quickly so a band aid does not have to be worn very long. However, even in these situations, it is recommended that the band aid be frequently changed to insure that the injury does not get infected and the area around the injury be kept clean.

Band aids usually have an outer surface which is “flesh” colored so as to blend in with the wearer's skin and not provide an unsightly appearance. However, people can usually tell at a glance when a band aid is being worn. Other band aids, particularly those worn by children, have an outer surface on which is printed a cartoon character or fanciful pattern. These band aids are usually not, however, worn by adolescents, teenagers, and adults.

### BRIEF SUMMARY OF THE INVENTION

The present invention is directed to an appliqué which is sized to fit over a band aid. As such, the appliqué is available is sizes and shapes corresponding to those of band aids. The appliqué has an adhesive backing which allows the band aid wearer to secure it over the top of the band aid. The outer surface of the appliqué has one of a number of decorative patterns formed on it. This not only gives the band aid a pleasing appearance, but allows the wearer, especially women, to now treat the band aid as an accessory by allowing them to co-ordinate the appliqué with the clothing they are wearing. Further, the appliqué has removable layers of adhesive material so the appliqué can be used repeatedly, as when the band aid is changed.

In addition to band aids, the appliqués can also be made for use with other types of bandages such as eye patches.

### BRIEF DESCRIPTION OF THE SEVERAL VIEWS OF THE DRAWINGS

The objects of the invention are achieved as set forth in the illustrative embodiments shown in the drawings which form a part of the specification.

FIGS. 1A and 1B illustrate band aids of different sizes and shapes;

FIG. 2A is plan view of appliqués of the present invention which fits over the top of a band aid to give it a decorative appearance, and FIG. 2B is an elevation view of the appliqués:

FIG. 3 illustrates the band aids of FIGS. 1A and 1B with the appliqués applied over the outer layer of the band aids;

FIG. 4 is a front elevation view of an eye patch;

FIG. 5A is a plan view of an appliqué of the present invention which fits over the eye patch to give it a decorative appearance, and FIG. 5B is an elevation view of the appliqué; and,

FIG. 6 illustrates the eye patch of FIG. 4 with the appliqué applied.

Corresponding reference characters indicate corresponding parts throughout the several views of the drawings.

### DETAILED DESCRIPTION OF INVENTION

The following detailed description illustrates the invention by way of example and not by way of limitation. This description will clearly enable one skilled in the art to make and use the invention, and describes several embodiments, adaptations, variations, alternatives and uses of the invention, including what I presently believe is the best mode of carrying out the invention. As various changes could be made in the above constructions without departing from the scope of the invention, it is intended that all matter contained in the above description or shown in the accompanying drawings shall be interpreted as illustrative and not in a limiting sense.

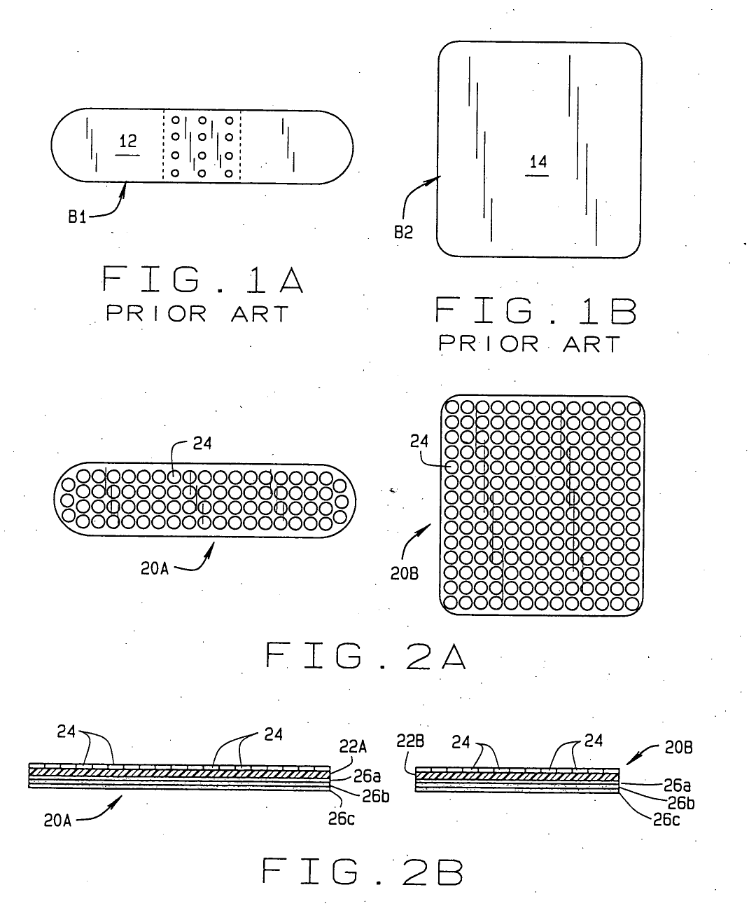

Referring to the drawings, conventional, prior art band aids are indicated generally B1 and B2 in FIGS. 1A and 1B. Band aid B1 is generally rectangular in shape and is used to cover and protect small cuts and scrapes. Band aid B2 is generally square in shape and is used to cover and protect larger cuts and scrapes. Each band aid has a top cover 12 and 14 respectively which comprises an outer layer of the bandage. It will be understood that band aids come in a variety of other sizes and shapes and that band aids B1 and B2 are representative only.

An appliqué of the present invention is indicated generally 20. Two versions of the appliqué are shown. One version 20A is for use with band aid B1 and a second version 20B is for use with band aid B2. As such, the length and width dimensions of the appliqués correspond to those of the band aids with which they are used. Again, the appliqués shown in the drawings are representative only and it will be appreciated that the appliqués are available in different sizes and shapes for different size band aids. Each appliqué has an outer layer 22A, 22B respectively. This top surface of this outer layer can be decorated in a wide range of ways. In the drawings, the layers have round colored discs 24 secured to them. However, the discs are exemplary only and those skilled in the art will understand that other materials can be used and that various designs, both arbitrary and fanciful can be used. For example, designs corresponding to various types of tattoos could be used.

Next, each appliqué has a number of layers 26 of an adhesive material attached to the underside of outer layer 22A, 22B. In the drawings, three layers 26a-26c are shown; although it will be understood there could be more or fewer layers. By having multiple adhesive layers, the appliqué can be reused rather than having to be discarded after a single use. If a band aid needs to be replaced fewer times than the number of adhesive layers, the appliqué can be returned to its package for subsequent use if the wearer requires.

In FIGS. 3A and 3B, the band aids B1 and B2 with the respective appliqués 20A and 20B applied are shown. It will be understood that wearing of a ban aid with an appliqué does not have to be only when the wearer, is injured. A person could, in effect, wear the band aid with appliqué as ornamentation similar to jewelry.

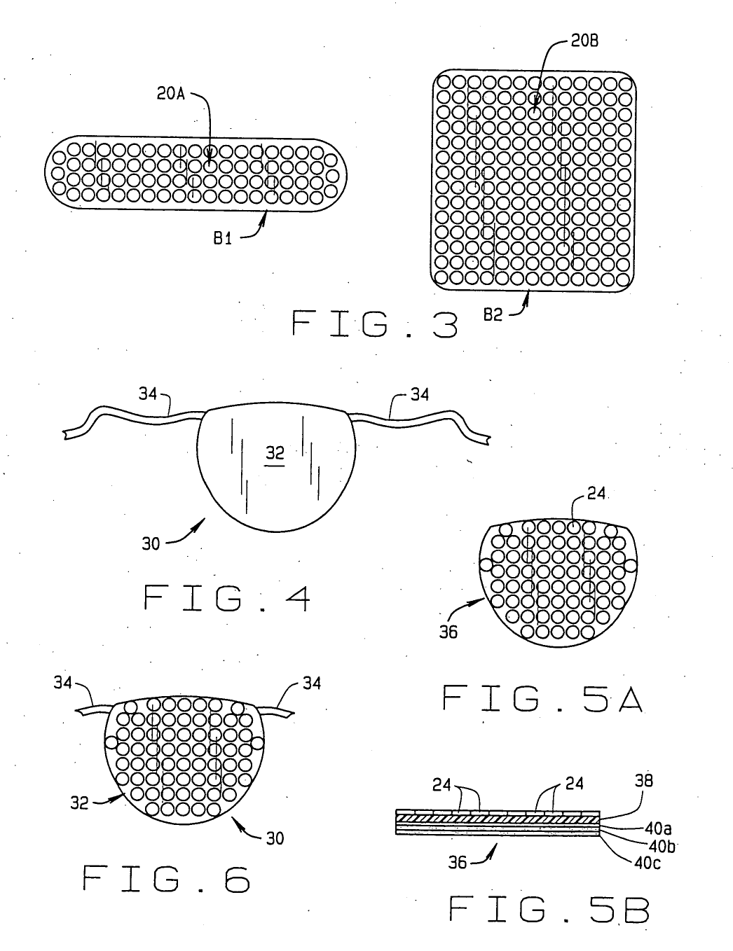

The appliqué of the present invention is not for use only with band aids. They can be used with other medical devices worn by a person. In FIG. 4, an eye patch indicated generally 30 has both a patch 32 and a ribbon 34 which is tied about the wearer's head to keep the eye patch in place.

In FIGS. 5A and 5B, an appliqué 36 is shown to be similar in shape to patch 32. The appliqué is decorated the same as appliqués 20A and 20B; although, again, other decorative arrangements are possible within the scope of the invention. The appliqué has an outer layer 38 with the round colored discs 24 secured to its top surface. The appliqué further has a number of layers 40 of adhesive material attached to the underside of the outer layer, three layers 40a-40c being shown in FIG. 5B. Finally, FIG. 6 illustrates the eye patch with the appliqué attached.

Those skilled in the art will understand that variety of other applications are possible. For example, the patches worn behind the ear by passengers on cruise ships.

In view of the above, it will be seen that the several objects and advantages of the present invention have been achieved and other advantageous results have been obtained.

1. An appliqué for attachment to the top layer of a band aid to provide a decorative appearance to the band aid when it is worn comprising:
a outer layer on a top surface of which a decorative material in a predetermined pattern is applied; and,
at least one layer of an adhesive material attached to the underside of the outer layer for attaching the appliqué to an outer surface of the band aid.

2. The appliqué of claim 1 having a plurality of layers of adhesive material so the appliqué can be removed from one band aid when it is discarded and attached to a new band aid put on in its place.

3. The appliqué of claim 1 which is the same size and shape as the band aid.

4. An appliqué for attachment to an eye patch to provide a decorative appearance to the patch when it is worn comprising:
a outer layer on a top surface of which a decorative material in a predetermined pattern is applied; and,
at least one layer of an adhesive material attached to the underside of the outer layer for attaching the appliqué to an outer surface of the patch.

5. The appliqué of claim 4 having a plurality of layers of adhesive material so the appliqué can be removed from the eye patch and reused when the eye patch is next worn.

6. The appliqué of claim 4 which is the same size and shape as the eye patch.
